(19) United States
(12) Patent Application Publication
(10) Pub. No.: US 2006/0173361 A1
(43) Pub. Date: Aug. 3, 2006

(54) Endoscopy capsule with site marking capability and application of the same

(75) Inventor: David Lee Gorden, Nashville, TN (US)

Correspondence Address: MORRIS MANNING & MARTIN LLP, 1600 ATLANTA FINANCIAL CENTER, 3343 PEACHTREE ROAD, NE, ATLANTA, GA, 30326-1044, US

(73) Assignee: Vanderbilt University, Nashville, TN (US)

(21) Appl. No.: 11/200,532

(22) Filed: Aug. 9, 2005

Related U.S. Application Data
(60) Provisional application No. 60/603,178, filed on Aug. 20, 2004.

(51) Int. Cl. A61B 6/00

(52) U.S. Cl. 600/478; 600/407; 600/476

(57) ABSTRACT

A capsule dimensioned and shaped to move with the body of a living subject for identifying tissues related to at least one lesion along a gastrointestinal tract of the living subject. In one embodiment, the capsule has locating means for locating tissues related to at least one lesion along the gastrointestinal tract, and marking means for marking the tissues related to at least one lesion along the gastrointestinal tract with an identification material stored within the capsule, wherein the marking means is automatically triggered when a predetermined condition is satisfied.

### CROSS-REFERENCE TO RELATED PATENT APPLICATION

This application claims the benefit, pursuant to 35 U.S.C. §119(e), of provisional U.S. patent application Ser. No. 60/603,178, filed Aug. 20, 2004, entitled “ENDOSCOPY CAPSULE WITH SITE MARKING CAPABILITY AND APPLICATION OF SAME,” by David Lee Gorden, which is incorporated herein by reference in its entirety.

Some references, which may include patents, patent applications and various publications, are cited and discussed in the description of this invention. The citation and/or discussion of such references is provided merely to clarify the description of the present invention and is not an admission that any such reference is “prior art” to the invention described herein. All references cited and discussed in this specification are incorporated herein by reference in their entireties and to the same extent as if each reference was individually incorporated by reference.

### BACKGROUND OF THE INVENTION

The most common diagnostic procedure for colonic examination is colonoscopy. This procedure involves the optical examination of the entire colon using a device known as a colonoscope. A colonoscope comprises a flexible tube containing a fiber optic imaging and illuminating device and a device to resect portions of the surface of the intestinal tract. The colonoscope is inserted into the rectum and can be maneuvered to the ileocecal junction (the start of the colon). The operator views the image on a video display. An sigmoidoscope is similar to a colonoscope, but can only be used to image the lower ⅔ portions of the colon. An esophagogastroduodenoscope is used to image the upper gastrointestinal tract, namely, the esophagus, the stomach and the duodenum. All these devices are inserted into a patient's tract through the mouth.

However, these devices see limited use for several reasons. First, they are invasive and uncomfortable to the patient, requiring sedation so that a flexible fiberoptic tube can be inserted into the tract. Secondly, these devices are expensive, requiring the presence of a highly trained physician and other personnel in operation. Third, they are often inconvenient, requiring the patient to take a purgative, fast overnight, and remain incapacitated during the procedure.

Various capsule endoscopies have been developed for overcoming the drawbacks. Different capsules have been utilized for mapping, marking, diagnosing and/or treating the intestinal tract by passing such capsules through the entire endoluminal gastrointestinal tract from “mouth to anus.” Basically, these capsules are small pill-like devices that can be ingested or swallowed by a patient. The capsules may have a sensor to determine a physiological parameter of the gastrointestinal tract. Some devices contemplate that the parameter be sensed and transmitted by an RF signal to an extra-corporeal antenna or receiver. For example, it is understood that U.S. Pat. No. 3,739,279 to Hollis discloses an oscillator circuit for such an ingestible capsule. The oscillator circuit has a type of Colpitts oscillator which may be used in association with a telemetry sensor for determining physiological information within the patient's body.

It is understood that U.S. Pat. No. 6,240,312 to Alfano et al. discloses an ingestible capsule that can be used to transmit a video signal to a location outside the body. However, the values of such sensed parameters may not be sufficient in and of themselves. Indeed, it is also important to know the location of the ingestible capsule at the time the physiological parameter is sensed. It us understood that U.S. Pat. No. 5,279,607 to Schentag et al. (“the '607 patent”) discloses an ingestible telemetry capsule with a means to determine location of the capsule. It is further understood that the '607 patent also discloses the capsule as having a medicament dispensing function.

Moreover, it is understood that U.S. Patent Application Publication 20040050394 by Jin (“the '394 publication”) discloses a system for moving a capsule within the body of an animal or human beings. The components of the system comprise a capsule dimensioned and shaped to move within the body of the animal. An anisotropic magnetic component is mechanically coupled to the capsule to move or orient the capsule in relation to an applied magnetic field, and a magnetic field generating system external of the body is provided for generating a three dimensionally oriented magnetic field within the body to move or orient the capsule.

It is understood that U.S. Patent Application Publication 20030167000 by Mullick et al. (“the '000 publication”) discloses a miniature ingestible capsule that has multiple therapeutic or diagnostic operations that can be performed. In the '000 publication, an imaging device A includes a capsule including an anterior membrane through which images are viewed, a lens positioned within the membrane, an illumination device (comprising a light source and projection device) positioned adjacent to the lens, an imaging array, transmitter, a pose beacon, a power source, and a posterior membrane.

It is understood that U.S. Patent Application Publication 20030040685 by Lewkowicz et al. (“the '685 publication”) discloses an in vivo examining device and method. According to the '685 publication, it is understood that the in vivo examining device has two operational phases: an initial phase in which the device is of initial dimensions and a final phase in which the device is of final dimensions.

It is understood that U.S. Patent Application Publication 20040068204 by Imran et al. (“the '204 publication”) discloses a device and method for mapping, diagnosing and treating the intestinal tract by using a capsule passing through the intestinal tract.

It is understood that U.S. Patent Application Publication 20030191430 by D'Andrea et al. (“the '430 publication”) discloses an improved ingestible capsule that is arranged to sense one or more physiological parameters within a mammalian body, and to transmit such parameters to an extra-corporeal receiver. In the '430 publication, it is understood that in use, the capsule and receiver perform the method of determining the real-time location of the capsule within a tract of a mammal.

Nevertheless, no one discloses or suggests a device and methods that are capable of identifying potential trouble spots on the endoluminal gastrointestinal tract and marking the identified spots for further and better medical treatment.

Therefore, a heretofore unaddressed need exists in the art to address the aforementioned deficiencies and inadequacies.

### SUMMARY OF THE INVENTION

In one aspect, the present invention relates to a method of identifying tissues related to at least one lesion along a gastrointestinal tract of a living subject. In one embodiment, the method includes the steps of providing a capsule for ingestion, locating tissues related to at least one lesion along the gastrointestinal tract, and marking the tissues related to at least one lesion along the gastrointestinal tract with an identification material stored within the capsule. The marking step, in one embodiment, includes the step of remotely releasing the identification material. In another embodiment, the marking step includes the step of releasing the identification material when a predetermined condition is satisfied, e.g., when the capsule contacts with hemoglobin.

The method further includes the step of illuminating the gastrointestinal tract via a light source within the capsule. The step of illuminating the gastrointestinal tract is performed prior to the step of locating tissues related to at least one lesion along the gastrointestinal tract, and after the step of marking the tissues related to at least one lesion along the gastrointestinal tract with an identification material stored within the capsule, respectively.

Moreover, the method includes the step of identifying the tissues marked with the identification material. Additionally, the method includes the step of performing a medical procedure related to the tissues marked with the identification material.

The capsule, in one embodiment, has a sub-capsule for storing at least one identification material, where the at least one identification material includes a dye. The dye comprises at least one of methelyne blue, India ink, or the like.

In another aspect, the present invention relates to a capsule dimensioned and shaped to move with the body of a living subject for identifying tissues related to at least one lesion along a gastrointestinal tract of the living subject. In one embodiment, the capsule has a chamber and a sub-capsule formed within the chamber, wherein the sub-capsule is in communication with an opening formed on the chamber wall of the capsule and isolated from the chamber. Furthermore, the capsule has an identification material stored within the sub-capsule and a releasing mechanism for selectively releasing the identification material for marking the tissues related to at least one lesion along the gastrointestinal tract of the living subject.

In one embodiment, the releasing mechanism is remotely controllable. The releasing mechanism includes a shutter that is movable between a first position and a second position. When the shutter is in the first position, the identification material is releasable to mark the tissues through the opening formed on the chamber wall. And when the shutter is in the second position, the identification material is non-releasable to mark the tissues through the opening formed on the chamber wall. In another embodiment, the releasing mechanism has a substance sensitive to a predetermined condition. The predetermined condition includes hemoglobin, abnormal tissue growth, or the like. For example, if hemoglobin is chosen as the predetermined condition, when the capsule contacts with hemoglobin, the identification material is releasable to mark the tissues related to hemoglobin through the opening formed on the chamber wall. In an alternative embodiment, the releasing mechanism includes a biosensor.

In yet another aspect, the present invention relates to a capsule dimensioned and shaped to move with the body of a living subject for identifying tissues along a gastrointestinal tract of the living subject. In one embodiment, the capsule has the locating means for locating tissues along the gastrointestinal tract and marking means for marking the tissues along the gastrointestinal tract with an identification material stored within the capsule. Furthermore, the capsule has a storing means within the capsule for storing at least one identification material. The marking means, in one embodiment, is remotely controllable. In another embodiment, the marking means is automatically triggered when a predetermined condition is satisfied.

These and other aspects of the present invention will become apparent from the following description of the preferred embodiment taken in conjunction with the following drawings, although variations and modifications therein may be affected without departing from the spirit and scope of the novel concepts of the disclosure.

### BRIEF DESCRIPTION OF THE DRAWINGS

FIG. 1 schematically shows a capsule navigating in a body of a living subject according to one embodiment of the present invention.

FIG. 2 schematically shows the capsule as shown in FIG. 1 releasing an identification material to mark tissues of lesion.

FIG. 3 schematically shows a side view of a capsule according to one embodiment of the present invention.

FIG. 4 schematically shows a side view of a capsule according to another embodiment of the present invention.

FIG. 5 schematically shows a perspective view of a capsule according to an alternative embodiment of the present invention.

### DETAILED DESCRIPTION OF THE INVENTION

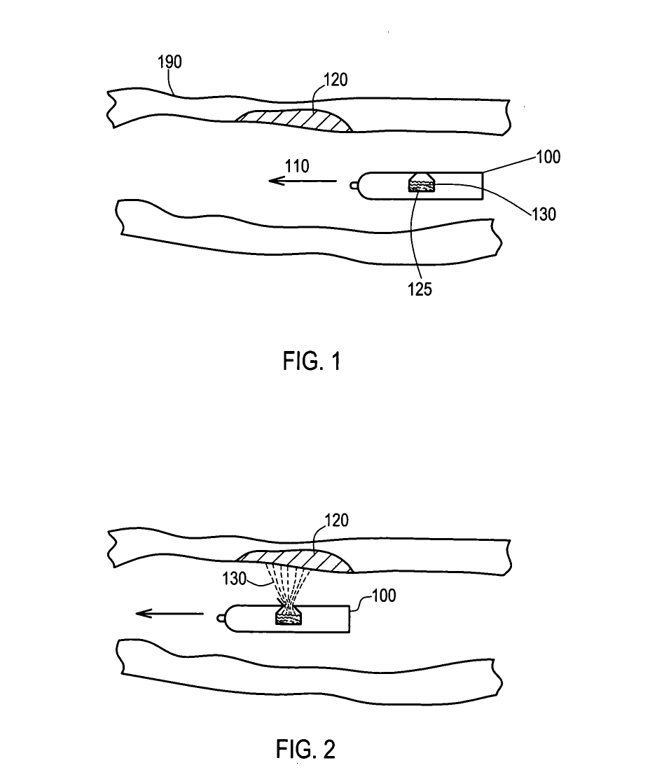

The present invention is more particularly described in the following examples that are intended as illustrative only since numerous modifications and variations therein will be apparent to those skilled in the art. As used in the specification and in the claims, “a” can mean one or more, depending upon the context in which it is used. Several embodiments are now described with reference to the FIGS. 1-5, in which like numbers indicate like parts throughout the FIGS. 1-5.

In one aspect, the present invention relates to a method of identifying tissues related to at least one lesion along a gastrointestinal tract of a living subject, where the living subject can be either a human beings or an animal. In one embodiment, the method includes the following steps: at first, a capsule for ingestion is provided, second, tissues related to at least one lesion along the gastrointestinal tract is located, and then the tissues related to at least one lesion along the gastrointestinal tract is marked with an identification material stored within the capsule. The marking step, in one embodiment, includes the step of remotely releasing the identification material. In another embodiment, the marking step includes the step of releasing the identification material when a predetermined condition is satisfied such as when the capsule contacts with hemoglobin.

The method further includes the step of illuminating the gastrointestinal tract via a light source within the capsule. The step of illuminating the gastrointestinal tract is performed prior to the step of locating tissues related to at least one lesion along the gastrointestinal tract, and after the step of marking the tissues related to at least one lesion along the gastrointestinal tract with an identification material stored within the capsule, respectively.

Additionally, the method includes the steps of identifying the tissues marked with the identification material, and performing a medical procedure related to the tissues marked with the identification material.

Referring now to FIGS. 1-5, and first to FIG. 1, as an example of but not limitations to the present invention, a capsule 100 is shown to be dimensioned and shaped to navigate in the body of a living subject, along a gastrointestinal tract 190, in a direction 110 for identifying tissues of lesion 120 along the gastrointestinal tract 190 of the living subject. Lesion 120 may be located anywhere along the gastrointestinal tract 190. The capsule 100 has an identification material 130 stored therein in a sub-capsule 125. In one embodiment, the identification material includes a dye. The dye has at least one of methelyne blue, India ink, a substance sensitive to a predetermined condition or the like. As shown in FIG. 2, when the capsule 100 moves to a location of the tissues of lesion 120, the identification material 130 will be released to the tissues of lesion 120 so as to mark the tissues of lesion 120. The release of the identification material 130 from the capsule 100 can be remotely controlled or locally triggered by a predetermined condition of the surrounding environment of the capsule 100.

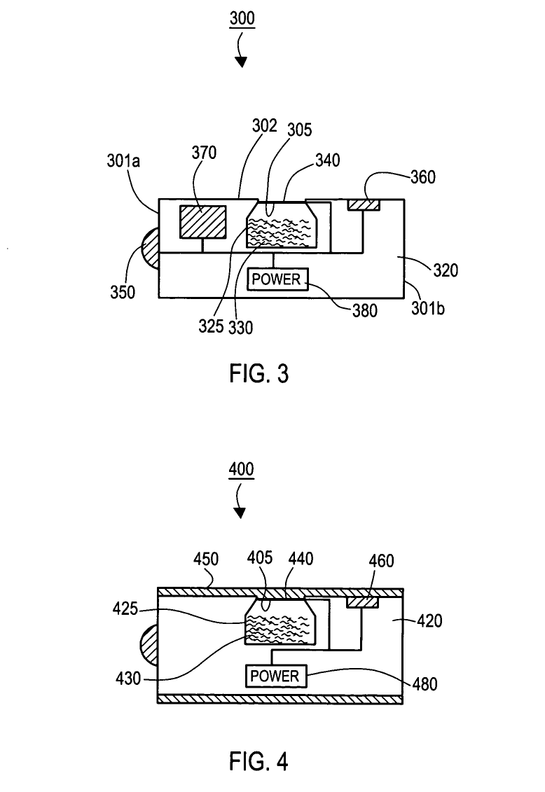

Referring to FIG. 3, a capsule 300, according to another embodiment as an example of but not limitations to the present invention, has a first end 301a, an opposite second end 301b, a body 302 defined therebetween the first end 301a and the opposite, second end 301b, and a chamber 320 formed therein the body 302. The capsule 300 further includes a light 350 on the first end 301a of the capsule 300 for illuminating a gastrointestinal tract of a living subject when navigating in the gastrointestinal tract. The capsule 300 also has a camera 370 placed in the chamber 320 for obtaining images of the gastrointestinal tract so as to determine conditions of the gastrointestinal tract, and a sub-capsule 325 formed therein the chamber 320 for storing an identification material 330, such as a dye. The images of the gastrointestinal tract obtained by the camera 370 can be transmitted, wirelessly or by the help of cable (not shown), to a monitoring device (not shown) in ways as known to people skilled in the art. The sub-capsule 325 is in communication with an opening 305 formed on the chamber wall of the capsule 300. Upon recognition of a predetermined condition such as abnormal tissue growth, an area of pathology and/or bleeding on the images obtained by the camera 370, the sub-capsule 325 containing the dye may be triggered remotely to release sufficient amount of the indentification material 330 to mark the area of pathology. A low energy rupture of the dye contained in the capsule can mark the tissues along the gastrointestinal tract such as bowel without adverse side effects.

Moreover, the capsule 300 has a releasing mechanism for selectively releasing the identification material 330 for marking tissues of lesion or tissues of interest along the gastrointestinal tract of the living subject. In one embodiment, the releasing mechanism includes a shutter 340 that is movable between a first position and a second position. When the shutter 340 is in the first position, the identification material 330 is releasable to mark the tissues through the opening 305 formed on the chamber wall as shown in FIG. 2. And when the shutter is in the second position, the identification material 330 is non-releasable to mark the tissues through the opening 305 formed on the chamber wall as shown in FIG. 1. The capsule 300 may include a micro-electro-mechanical system (hereinafter “MEMS”) 360 in communications with the camera 370 and the shutter 340 for controlling the release of the identification material 330. The MEMS 360 is bio-compatible. In one embodiment, the MEMS 360 can be programmed so as to be remotely controllable. Additionally, the capsule 300 has a power supply 380 for supplying power to the light 350, the camera 270, the shutter 340 and the MEMS 360, respectively. Power supply can also be provided through a cable (not shown) in ways as known to people skilled in the art.

FIG. 4 shows a capsule 400 according to another embodiment of the present invention. The capsule 400 has a chamber 420, a sub-capsule 425 formed therein the chamber 420 for storing an identification material 430, a shutter 440 placed on an opening 405 of a chamber wall in communication with the sub-capsule 425 for releasing an identification material 430 such as a dye, a substance 450 coated on an outer surface of the chamber wall for sensing a predetermined condition of the surrounding environment of the capsule 400 and a biosensor 460 placed inside the chamber 420 in communication with the shutter 440 and the coated substance 450 for controlling the release of the identification material 330. The substance 450 is sensitive to the predetermined condition, such as hemoglobin, such that when the capsule 400 contacts with hemoglobin, a signal is triggered and received by the biosensor 460, which then communicates with the shutter 440 to release the identification material 430 to the bleeding area so as to mark the bleeding tissues. The substance 450 will not be stained by the dye or disrupt endoluminal visualization. Additionally, the capsule 400 has a power supply 480 within the chamber 420 for supplying power to the shutter 440 and the biosensor 460, respectively.

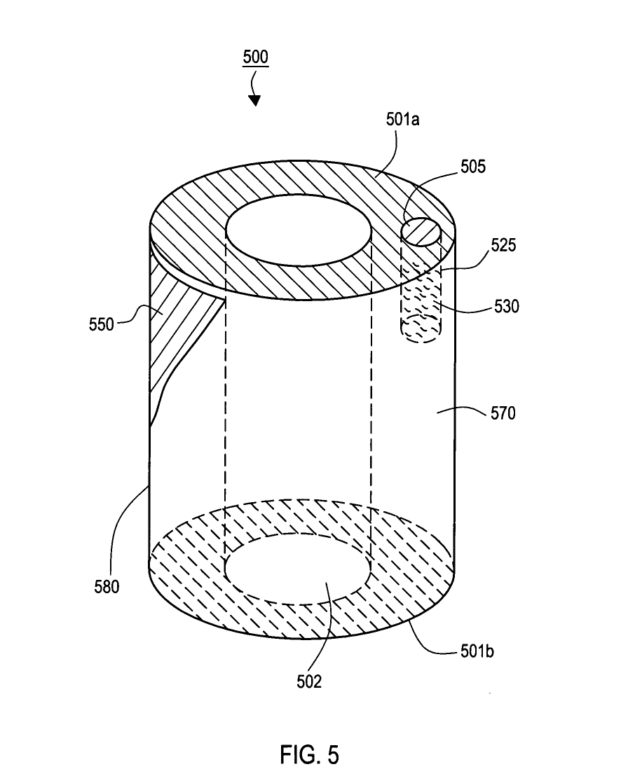

Referring to FIG. 5, a capsule 500 is shown according to an alternative embodiment of the present invention. The capsule 500 has a first end 501a, an opposite, second end 501b, a hollow body 502 defined therebetween the first end 501a and the opposite, second end 501b. The capsule 500 further has a sub-capsule 525 placed inside a body wall 570 of the capsule 500 and isolated from the hollow body 502. The sub-capsule 525 is in communication with an opening 505 of the first end 501a of the capsule 500. Moreover, the capsule 500 has an identification material 530 stored in the sub-capsule 525 and a bio-sensitive coating area 550 on the outer surface 580 around the first end portion 501a of the capsule 500. The bio-sensitive coating area 550 is coated with a substance sensitive to a predetermined condition of the surrounding environment of the capsule 500 such that when the predetermined condition is satisfied, the identification material 530 stored in the sub-capsule 525 will be automatically triggered to release to the surrounding environment through the opening 505 of the first end 501a of the capsule 500 to mark the area where the predetermined condition is satisfied. Alterenatively, the capsule 500 may also contain a camera (not shown) to capture images of the surrounding environment to allow an operator to mark any spot at his will.

Subsequent surgical exploration of the living subject such as a patient can be carried out in a timely fashion, which would allow a surgeon to see a discreet, marked tissues of interest such as segment of bowel and target the marked site for further treatment such as resection. Among other things, the present invention provides devices and methods that have an advantage of localizing the pathology and preventing unnecessary resection of bowel, and unnecessary morbidity to the patient. This precise localization of pathology has the potential to save many lives from the lift threatening consequences of gastrointestinal bleeding.

While there has been shown several and alternate embodiments of the present invention, it is to be understood that certain changes can be made as would be known to one skilled in the art without departing from the underlying scope of the invention as is discussed and set forth above and below. Furthermore, the embodiments described above are only intended to illustrate the principles of the present invention and are not intended to limit the scope of the invention to the disclosed embodiments.

What is claimed is:

1. A method of identifying tissues related to at least one lesion along a gastrointestinal tract of a living subject, comprising the steps of:
a. providing a capsule for ingestion;
b. locating tissues related to at least one lesion along the gastrointestinal tract; and
c. marking the tissues related to at least one lesion along the gastrointestinal tract with an identification material stored within the capsule.

2. The method of claim 1, wherein the capsule has a sub-capsule for storing at least one identification material.

3. The method of claim 2, wherein the identification material comprises a dye.

4. The method of claim 3, wherein the dye comprises at least one of methelyne blue, India ink, or the like.

5. The method of claim 1, wherein the marking step further comprises the step of remotely releasing the identification material.

6. The method of claim 1, wherein the marking step further comprises the step of releasing the identification material when the capsule contacts with hemoglobin.

7. The method of claim 1, further comprising the step of illuminating the gastrointestinal tract via a light source within the capsule.

8. The method of claim 7, wherein the step of illuminating the gastrointestinal tract is performed prior to the step of locating tissues related to at least one lesion along the gastrointestinal tract.

9. The method of claim 7, wherein the step of illuminating the gastrointestinal tract is performed after the step of marking the tissues related to at least one lesion along the gastrointestinal tract with an identification material stored within the capsule.

10. The method of claim 9, further comprising the step of identifying the tissues marked with the identification material.

11. The method of claim 10, further comprising the step of performing a medical procedure related to the tissues marked with the identification material.

12. A capsule dimensioned and shaped to move with the body of a living subject for identifying tissues related to at least one lesion along a gastrointestinal tract of the living subject, comprising:
a. a chamber;
b. a sub-capsule formed within the chamber, wherein the sub-capsule is in communication with an opening formed on the chamber wall of the capsule and isolated from the chamber;
c. an identification material stored within the sub-capsule; and
d. a releasing mechanism for selectively releasing the identification material for marking the tissues related to at least one lesion along the gastrointestinal tract of the living subject.

13. The capsule of claim 12, wherein the identification material comprises a dye.

14. The capsule of claim 13, wherein the dye comprises at least one of methelyne blue, India ink, or the like.

15. The capsule of claim 12, wherein the releasing mechanism is remotely controllable.

16. The capsule of claim 15, wherein the releasing mechanism comprises a shutter that is movable between a first position and a second position.

17. The capsule of claim 15, wherein when the shutter is in the first position, the identification material is releasable to mark the tissues through the opening formed on the chamber wall, and wherein when the shutter is in the second position, the identification material is non-releasable to mark the tissues through the opening formed on the chamber wall.

18. The capsule of claim 12, wherein the releasing mechanism comprises a substance sensitive to a predetermined condition.

19. The capsule of claim 18, wherein the predetermined condition comprises hemoglobin, and when the capsule contacts with hemoglobin, the identification material is releasable to mark the tissues related to the predetermined condition through the opening formed on the chamber wall.

20. The capsule of claim 12, wherein the releasing mechanism comprises a biosensor.

21. A capsule dimensioned and shaped to move with the body of a living subject for identifying tissues along a gastrointestinal tract of the living subject, comprising:
a. locating means for locating tissues along the gastrointestinal tract; and
b. marking means for marking the tissues along the gastrointestinal tract with an identification material stored within the capsule.

22. The capsule of claim 21, further comprising a storing means within the capsule for storing at least identification material.

23. The capsule of claim 21, wherein the identification material comprises a dye.

24. The capsule of claim 23, wherein the dye comprises at least one of methelyne blue, India ink, or the like.

25. The capsule of claim 21, wherein the marking means is remotely controllable.

26. The capsule of claim 21, wherein the marking means is automatically triggered when a predetermined condition is satisfied.
